(19) United States
(12) Patent Application Publication
(10) Pub. No.: US 2025/0120903 A1
(43) Pub. Date: Apr. 17, 2025

(54) NOVEL SPHINGOMONAS PAUCIMOBILIS STRAIN AND USE THEREOF

(71) Applicant: GENOME AND COMPANY, Seongnam-si Gyeonggi-do (KR)

(72) Inventors: Riwon HONG, Seongnam-si (KR); Hye Hee JEON, Seongnam-si (KR); Suekyoung YOON, Seongnam-si (KR); Jaesung YEON, Seongnam-si (KR); Shinyoung PARK, Seongnam-si (KR); Giduk BAE, Seongnam-si (KR)

(73) Assignee: GENOME AND COMPANY, Seongnam-si Gyeonggi-do (KR)

(21) Appl. No.: 18/285,157

(22) PCT Filed: Apr. 1, 2022
(86) PCT No.: PCT/KR2022/004737

§ 371 Date: Dec. 19, 2024

(30) Foreign Application Priority Data
Apr. 1, 2021 (KR) 10-2021-0043018

(51) Int. Cl. A61K 8/99; A61Q 19/00; A61Q 19/08; C12N 1/20; C12R 1/01

(52) U.S. Cl. CPC A61K 8/99; A61Q 19/007; A61Q 19/08; C12N 1/205; C12R 2001/01

(57) ABSTRACT

The present invention relates to a novel Sphingomonas paucimobilis strain. In addition, it relates to a use of Sphingomonas paucimobilis strain. Specifically, it relates to a method or composition for preventing or improving skin wrinkles, enhancing skin elasticity, reinforcing a skin barrier, or moisturizing the skin using the strain. The novel Sphingomonas paucimobilis strain according to the present invention can inhibit the expression of a collagen-degrading enzyme MMP-1, and promote the expression of collagen, filaggrin and claudin-1 that exhibit effects to improve skin wrinkles, enhance skin elasticity, strengthen skin barrier, and moisturize the skin. Therefore, the strain can be usefully used to prevent or improve skin wrinkles, enhance skin elasticity, strengthen the skin barrier, and/or moisturize the skin.

### TECHNICAL FIELD

The present invention relates to a novel Sphingomonas paucimobilis strain. In addition, it relates to a use of Sphingomonas paucimobilis strain. Specifically, it relates to a method or composition for preventing or improving skin wrinkles, enhancing skin elasticity, reinforcing a skin barrier, or moisturizing the skin using the strain.

### BACKGROUND ART

Skin aging, which is characterized by skin wrinkles, decreased skin elasticity, weakened skin barrier, and weakened moisturizing function, occurs by intrinsic and extrinsic factors. Aging caused by intrinsic factors is a natural phenomenon that occurs with age without specific environmental influences. On the other hand, aging caused by extrinsic factors is attributed to environmental elements, including ultraviolet radiation, smoking, stress, chemicals, and pollution. Among these, UV radiation is particularly considered a primary cause of aging.

During this skin aging process, major components of skin, such as lipids, proteins, polysaccharides, and nucleic acids, are oxidized, and skin cells and tissues are destroyed. In particular, when the proteins that make up skin connective tissue, such as collagen, filaggrin, and claudin-1, are damaged, an excessive inflammatory response occurs and the elasticity of the skin decreases.

Collagen, one of the skin's constituent proteins mentioned above, is a major matrix protein produced in skin fibroblasts, and plays a major role in strengthening the skin barrier such as providing mechanical rigidity of the skin, connective tissue resistance and tissue adhesion, supporting cell adhesion, and inducing cell division and differentiation during wound healing. It decreases with factors such as aging and photoaging caused by exposure to ultraviolet radiation. Additionally, the expression of matrix metalloproteinase-1 (MMP-1), an enzyme responsible for collagen degradation, increases due to the mentioned factors, potentially further accelerating skin aging through collagen decomposition. In other words, within the body, the synthesis and degradation of extracellular matrices such as collagen are appropriately regulated. However, as aging progresses, collagen synthesis decreases and the expression of MMP-1, a collagen-degrading enzyme, is promoted, leading to a reduction in skin elasticity and the formation of wrinkles. Therefore, in order to prevent skin aging and wrinkles, it is necessary to suppress collagen degradation and promote collagen production by inhibiting the expression and activity of MMP-1 in cells.

In addition, recent research results have reported that alterations in the filaggrin gene and abnormal expression of the claudin-1 gene are closely related to the onset and progression of atopic dermatitis and have a negative impact on the function and moisturization of the skin barrier. (Acta Derm Venereol. 2018 Jan 12, 98(1):19-25; An Bras Dermatol. Jul-Aug 2016, 91(4):472-8., etc.). Specifically, filaggrin is considered a natural moisturizing factor. When moisture decreases in the outer layer of the epidermis, filaggrin protein is hydrolyzed and broken down into hygroscopic amino acids, thereby maintaining moisture in the upper stratum corneum of the skin. In addition, claudin-1 is a protein that constitutes tight junctions that play a major role in strengthening the skin barrier and retaining moisture. Through tight junctions by claudin-1, transepidermal water loss (TEWL) is suppressed and the skin barrier is strengthened.

Therefore, in order to prevent or improve skin wrinkles and enhance the skin barrier and skin moisturizing function, it is necessary to inhibit the decomposition of collagen mediated by the body's metabolic process and ultraviolet irradiation, and promote the expression of related genes and/or proteins such as collagen, filaggrin or claudin-1. Substances currently known to be effective in improving skin wrinkles and moisturizing the skin include adenosine and retinoic acid (RA). However, adenosine has shown limited efficacy in clinical study, and retinoic acid cannot be used in fertile women and has side effects such as erythema. Therefore, there is still a need in the art for a cosmetic composition that can exhibit the above-mentioned skin improvement effect.

### TECHNICAL SOLUTION

The inventors conducted various studies on strains that can exhibit skin improvement effects. As a result, a new Sphingomonas paucimobilis GENSC03 was discovered and deposited, and it was experimentally proven that it exhibits excellent skin wrinkle improvement, skin elasticity enhancement, skin barrier strengthening, and skin moisturizing effects, thereby completing the present invention.

The present invention provides a novel Sphingomonas paucimobilis strain.

The term “Sphingomonas paucimobilis” refers to the Sphingomonas paucimobilis GENSC03 strain. In this specification, ‘Sphingomonas paucimobilis’ may be used interchangeably with ‘Sphingomonas paucimobilis strain’, ‘Sphingomonas paucimobilis GENSC03’, ‘Sphingomonas paucimobilis GENSC03 strain’, ‘GENSC03’ and ‘GENSC03 strain’. The strain is interpreted to include a strain itself, a culture of strain, a lysate of strain, an extract of strain, and a cytoplasmic fraction obtained by lysing the strain.

The term ‘culture’ refers to an entire medium including the strain itself, extracts of the strain, metabolites of the strain, or extra nutrients obtained by culturing the strain for a certain period of time on a medium supplying nutrients. It also includes a culture solution from which the strain was removed after culturing. Additionally, it may include a concentrate of the entire medium or culture solution, and a dried product of the concentrate. Specifically, the culture of the present invention can use a medium easily selected by a person skilled in the art depending on the desired purpose among the media used for microbial culture, for example, a medium used for Sphingomonas paucimobilis culture, such as Tryptic soy agar (TSA) or Tryptic soy broth (TSB) medium. However, it is not limited thereto, as long as Sphingomonas paucimobilis can be cultured.

The Sphingomonas paucimobilis GENSC03 was deposited by the applicant at the Korea Research Institute of Bioscience and Biotechnology under the accession number KCTC 14495BP on Mar. 11, 2021, and has the nucleotide sequence of the 16S rRNA gene represented by SEQ ID NO: 1.

Additionally, the Sphingomonas paucimobilis GENSC03 may reduce the expression or activity of MMP-1 (matrix metalloproteinase-1). Additionally, it may increase the expression or activity of one or more selected from the group consisting of collagen type 1, filaggrin, and claudin-1. When the expression or activity of MMP-1 is reduced, or the expression or activity of collagen type 1, filaggrin or claudin-1 is increased, the effects of preventing or improving skin wrinkles, maintaining or strengthening skin elasticity, maintaining or strengthening the skin barrier, and/or moisturizing the skin are obtained. Therefore, the Sphingomonas paucimobilis GENSC03 can be effectively used for preventing or improving skin wrinkles, enhancing skin elasticity, strengthening the skin barrier, or moisturizing the skin.

Another aspect of the present invention provides a use of one or more selected from the group consisting of a Sphingomonas paucimobilis strain, a culture of the strain, a fermentation product of the strain, a lysate of the strain, an extract of the strain, and a cytoplasmic fraction obtained by lysing the strain for preventing or improving skin wrinkles, enhancing skin elasticity, strengthening the skin barrier, or moisturizing the skin.

In the use of preventing or improving skin wrinkles, enhancing skin elasticity, strengthening the skin barrier, or moisturizing the skin according to the present invention, the related terms are understood to have the same meaning as those described above, unless specifically stated otherwise.

Another aspect of the present invention provides a cosmetic composition comprising one or more selected from the group consisting of a Sphingomonas paucimobilis strain, a culture of the strain, a fermentation product of the strain, a lysate of the strain, an extract of the strain, and a cytoplasmic fraction obtained by lysing the strain.

In the cosmetic composition according to the present invention, the related terms are understood to have the same meaning as those described above, unless specifically stated otherwise.

The cosmetic composition of the present invention comprises the Sphingomonas paucimobilis GENSC03 as an active ingredient. In addition, as described above, the Sphingomonas paucimobilis GENSC03 is interpreted to include a Sphingomonas paucimobilis GENSC03 strain itself, a culture of the strain, a fermentation product of the strain, a lysate of the strain, an extract of the strain, or a cytoplasmic fraction obtained by lysing the strain, and thus the cosmetic composition of the present invention comprises a Sphingomonas paucimobilis GENSC03 strain, a culture of the strain, a fermentation product of the strain, a lysate of the strain, an extract of the strain, or a cytoplasmic fraction obtained by lysing the strain.

The cosmetic composition of the present invention may be used to prevent or improve skin wrinkles, or to moisturize the skin. In addition, the effect of preventing or improving skin wrinkles may have the effect of maintaining or strengthening skin elasticity and/or barrier.

The content of Sphingomonas paucimobilis GENSC03 in the cosmetic composition may be appropriately determined considering various factors such as purpose of use, period of use, type of formulation, route of administration, and skin condition of the user.

Additionally, there are no restrictions on gender, age, etc. for users of the cosmetic composition. Specifically, anyone who wants to prevent or improve skin wrinkles or moisturize skin may use it.

In addition, the cosmetic composition may further comprise additional ingredients in addition to the Sphingomonas paucimobilis GENSC03. The additional ingredients are not limited as long as they do not offset or reduce the effect of preventing or improving skin wrinkles or moisturizing the skin, and may include, for example, conventional ingredients typically used to add or enhance cosmetic functions. Specifically, it may be one or more water-based additives selected from stabilizers, emulsifiers, thickeners, moisturizers, liquid crystal film strengtheners, pH adjusters, antibacterial agents, water-soluble polymers, coating agents, metal ion sequestrants, amino acids, organic amines, polymer emulsions, pH adjusters, skin nutrients, antioxidants, preservatives, and fragrances; or one or more oil-based additives selected from fat and oils, waxes, hydrocarbon oils, higher fatty acid oils, higher alcohols, synthetic ester oils, and silicone oils. In addition, it may further include acceptable carriers such as solvents, surfactants, suspending agents-nonsurfactants, moisturizers, opacifiers, anti-foaming agents, bulking agents, skin softeners, skin conditioning agents, chelating agents, preservatives, and sterilizing preservatives.

Additionally, the cosmetic composition may be prepared in the form of an usual emulsified formulation or solubilized formulation using commonly known manufacturing methods. Specifically, it can be manufactured in the various formulations such as patches, ointments, skin adhesive gels, creams, packs, tonics, essences, sprays, masks, foundations, makeup bases, cleansers, water (W) type, oil (O) type. silicone(S) type, oil-in-water (O/W) type, water-in-oil (W/O) type, water-in-silicon (W/S) type, silicone-in-water (S/W) type, solid, and liquid, and commonly used manufacturing methods may be applied.

Another aspect of the present invention provides a method for preventing or improving skin wrinkles, enhancing skin elasticity, strengthening the skin barrier, or moisturizing the skin, comprising the step of administering one or more selected from the group consisting of a Sphingomonas paucimobilis strain, a culture of the strain, a fermentation product of the strain, a lysate of the strain, an extract of the strain, and a cytoplasmic fraction obtained by lysing the strain.

In the method for preventing or improving skin wrinkles, enhancing skin elasticity, strengthening skin barrier, or moisturizing skin according to the present invention, the related terms are understood to have the same meaning as those described above, unless specifically stated otherwise.

Sphingomonas paucimobilis GENSC03 of the present invention may be administered in an effective amount to a subject in need of preventing or improving skin wrinkles, enhancing skin elasticity, strengthening the skin barrier, or moisturizing the skin.

As used herein, the term “administration” refers to the physical introduction of a composition into a subject using any of a variety of methods and delivery systems known to those skilled in the art. The administration may be performed, for example, by oral administration or transdermal administration, but not limited thereto. The number of administrations may be, for example, single, multiple, or over one or more extended periods.

As used herein, the term “subject” includes a human or any non-human animal, and the non-human animal may be a vertebrate such as a primate, dog, cow, horse, pig, rodent such as mouse, rat, and guinea pig. In this specification, “subject” is used interchangeably with “individual” and “patient”.

The effective amount may be the minimum amount capable of preventing or improving skin wrinkles, enhancing skin elasticity, strengthening the skin barrier, or moisturizing the skin, or the maximum amount capable of preventing or improving skin wrinkles, enhancing skin elasticity, strengthening the skin barrier, or moisturizing the skin without causing side effects or toxicity. The level of the effective amount may be determined depending on factors including the severity of the subject, age, gender, activity of the strain, sensitivity to the strain, time of administration, route of administration and excretion rate, duration of treatment, drugs used together, and other factors well known in the field.

In addition, the dosage of Sphingomonas paucimobilis GENSC03 may vary depending on the subject's age, gender, and weight. Specifically, depending on the subject's symptoms, 0.1 to 100 mg/kg of the strain may be administered once or several times a day, or at intervals of several days to several months. Additionally, the dosage may be increased or decreased depending on the route of administration, severity of symptoms, gender, weight, age, etc.

Additionally, the Sphingomonas paucimobilis GENSC03 may be administered in combination with other materials. In this case, the GENSC03 strain of the present invention and other materials may be administered simultaneously, sequentially, or separately. The other materials may be substances such as compounds, proteins, and extracts that have the effect of preventing or improving skin wrinkles, enhancing skin elasticity, strengthening the skin barrier, or moisturizing the skin, but are not limited thereto.

Additionally, the Sphingomonas paucimobilis GENSC03 may be formulated to be administered simultaneously, sequentially, or individually with other materials. For example, the strain and other materials may be administered simultaneously in one formulation, or may be administered simultaneously, sequentially, or separately in separate formulations. For the simultaneous, sequential or separate administration, the GENSC03 strain and other materials may be separately formulated in separate containers or may be formulated together in the same container. In addition, the effective dose, administration time, administration interval, administration route, treatment period, etc. of the GENSC03 strain and other materials may be the same or different each other.

Additionally, the Sphingomonas paucimobilis GENSC03 may be administered to a subject simultaneously, sequentially, or individually with other materials.

The “simultaneous” administration refers to administering the GENSC03 strain and other materials at once as one formulation, or administering the GENSC03 strain and other materials at once as separate formulations, wherein the administration route of the GENSC03 strain and other materials may be different. In addition, the “sequential” administration refers to administering the GENSC03 strain and other materials relatively continuously, allowing the minimum time possible as time spent between administrations. Additionally, the “separate” administration refers to administering the GENSC03 strain and other materials at regular time intervals. The method of administering the GENSC03 strain and other materials may be appropriately selected by a doctor or expert in the art considering the treatment efficacy and side effects of the subject.

The new Sphingomonas paucimobilis strain according to the present invention can inhibit the expression of MMP-1, a collagen-degrading enzyme, and promote the expression of collagen, filaggrin, and claudin-1 that exhibit effects to improve skin wrinkles, enhance skin elasticity, strengthen skin barrier, or moisturize the skin.

Therefore, the strain can be usefully used to prevent or improve skin wrinkles, enhance skin elasticity, strengthen the skin barrier, and/or moisturize the skin.

### BRIEF DESCRIPTION OF FIGURES

FIG. 1 is an image showing the culture results of Sphingomonas paucimobilis GENSC03 cultured on blood agar medium.

FIG. 2 is a graph showing the cell viability of human dermal fibroblasts treated with a culture of Sphingomonas paucimobilis GENSC03 strain. ‘TSB’ refers to the control group treated with TSB (Tryptic Soy Broth) liquid medium, and ‘GENSC03’ refers to the experimental group treated with the culture of GENSC03 strain.

FIG. 3 is a graph showing the expression level of matrix metalloproteinase-1 (MMP-1) in human dermal fibroblasts treated with a culture of the Sphingomonas paucimobilis GENSC03 strain. ‘TSB’ refers to the control group treated with TSB liquid medium, ‘RA’ refers to the positive control group treated with retinoic acid (1 μM), and ‘GENSC03’ refers to the experimental group treated with the culture of GENSC03 strain.

FIG. 4 is a graph showing the expression level of collagen type 1 in human dermal fibroblasts treated with a culture of Sphingomonas paucimobilis GENSC03 strain. ‘TSB’ refers to the control group treated with TSB liquid medium, ‘RA’ refers to the positive control group treated with retinoic acid (1 μM), and ‘GENSC03’ refers to the experimental group treated with the culture of GENSC03 strain.

FIG. 5 is a graph showing the level of filaggrin mRNA expression in human keratinocyte (HaCaT) treated with a culture of the Sphingomonas paucimobilis GENSC03 strain. The mRNA expression level of filaggrin was expressed as a value normalized to the mRNA expression level of β-actin. ‘TSB’ refers to a control group treated with TSB liquid medium, ‘RA’refers to a positive control group treated with retinoic acid (1 μM), and ‘24H’ and ‘36H’ refer to the experimental group treated with the culture of GENSC03 strain (5%) for 24 hours and 36 hours, respectively.

FIG. 6 is a graph showing the level of claudin-1 mRNA expression in human keratinocyte (HaCaT) treated with a culture of the Sphingomonas paucimobilis GENSC03 strain. The mRNA expression level of claudin-1 was expressed as a value normalized to the mRNA expression level of β-actin. ‘TSB’ refers to a control group treated with TSB liquid medium, ‘RA’refers to a positive control group treated with retinoic acid (1 μM), and ‘24H’ and ‘36H’ refer to the experimental group treated with the culture of GENSC03 strain (5%) for 24 hours and 36 hours, respectively.

### EXAMPLES

Hereinafter, the present invention will be described in more detail through examples. These examples are for illustrating the present invention in more detail, and the scope of the present invention is not limited by these examples.

### Example 1. Isolation and Identification of Sphingomonas paucimobilis GENSC03

In order to confirm strains that prevent or improve skin wrinkles, enhance skin elasticity, strengthen the skin barrier, and/or moisturize the skin, we isolated bacteria derived from the skin of adults who had never suffered from skin diseases such as atopy, psoriasis, or acne, or had not received related treatment within the past 6 months.

Specifically, the unwashed cheeks and the bridge of the nose were vigorously rubbed with a sterilized cotton swab soaked in sterile water. Thereafter, the swab with the skin sample was placed in a tube containing PBS and sealed, and then smeared on Tryptic soy agar (TSA) to isolate strains. This operation was repeated 3 to 4 times to isolate pure colonies.

Afterwards, the nucleotide sequence of 16S rRNA gene was determined from the pure culture of the isolated strain to identify the strain. The used primer sequences and 16S rRNA gene nucleotide sequence of the isolated strain are shown in Table 1 below.

TABLE 1

| Type | Sequence | SEQ ID NO. |
|---|---|---|
| 16S rRNA gene | CTGTCAGAACGAACGCTGGCGGCATGCCTAAC | SEQ ID NO: 1 |
| | ACATGCAAGTCGAACGAAGGCTTCGGCCTTAG | |
| | TGGCGCACGGGTGCGTAACGCGTGGGAATCTG | |
| | CCCTTAGGTTCGGAATAACAGCTGGAAACGGC | |
| | TGCTAATACCGGATGATATCGCGAGATCAAAG | |
| | ATTTATCGCCTGAGGATGAGCCCGCGTTGGAT | |
| | TAGGTAGTTGGTGGGGTAAAGGCCTACCAAGC | |
| | CGACGATCCATAGCTGGTCTGAGAGGATGATC | |
| | AGCCACACTGGGACTGAGACACGGCCCAGAC | |
| | TCCTACGGGAGGCAGCAGTGGGGAATATTGG | |
| | ACAATGGGCGAAAGCCTGATCCAGCAATGCC | |
| | GCGTGAGTGATGAAGGCCCTAGGGTTGTAAA | |
| | GCTCTTTTACCCGGGAAGATAATGACTGTACC | |
| | GGGAGAATAAGCCCCGGCTAACTCCGTGCCA | |
| | GCAGCCGCGGTAATACGGAGGGGGCTAGCGT | |
| | TGTTCGGAATTACTGGGCGTAAAGCGCACGTA | |
| | GGCGGCTTTGTAAGTCAGAGGTGAAAGCCTGG | |
| | AGCTCAACTCCAGAACTGCCTTTGAGACTGCA | |
| | TCGCTTGAATCCAGGAGAGGTCAGTGGAATTC | |
| | CGAGTGTAGAGGTGAAATTCGTAGATATTCGG | |
| | AAGAACACCAGTGGCGAAGGCGGCTGACTGG | |
| | ACTGGTATTGACGCTGAGGTGCGAAAGCGTGG | |
| | GGAGCAAACAGGATTAGATACCCTGGTAGTCC | |
| | ACGCCGTAAACGATGATAACTAGCTGTCCGGG | |
| | CACTTGGTGCTTGGGTGGCGCAGCTAACGCAT | |
| | TAAGTTATCCGCCTGGGGAGTACGGCCGCAAG | |
| | GTTAAAACTCAAAGGAATTGACGGGGGCCTG | |
| | CACAAGCGGTGGAGCATGTGGTTTAATTCGAA | |
| | GCAACGCGCAGAACCTTACCAGCGTTTGACAT | |
| | GGTAGGACGACTTCCAGAGATGGATTTCTTCC | |
| | CTTCGGGGACCTACACACAGGTGCTGCATGGC | |
| | TGTCGTCAGCTCGTGTCGTGAGATGTTGGGTT | |
| | AAGTCCCGCAACGAGCGCAACCCTCGCCTTTA | |
| | GTTACCATCATTTGGTTGGGTACTCTAAAGGA | |
| | ACCGCCGGTGATAAGCCGGAGGAAGGTGGGG | |
| | ATGACGTCAAGTCCTCATGGCCCTTACGCGCT | |
| | GGGCTACACACGTGCTACAATGGCAACTACAG | |
| | TGGGCAGCGACCCTGCGAGGGCGAGCTAATC | |
| | CCCAAAAGTTGTCTCAGTTCGGATTGTTCTCTG | |
| | CAACTCGAGAGCATGAAGGCGGAATCGCTAG | |
| | TAATCGCGGATCAGCATGCCGCGGTGAATACG | |
| | TTCCCAGGCCTTGTACACACCGCCCGTCACAC | |
| | CATGGGAGTTGGATTCACCCGAAGGCGTTGCG | |
| | CCAACCTAGCAATAGGAAGCAGGCGACCACG | |
| | GTGGGTTCAGCGACTGGGGTGAAGC | |
| Forward Primer | 5′-AGAGTTTGATCMTGGCTCAG-3′ | SEQ ID NO: 2 |
| 27F (used for | | |
| PCR) | | |
| Reverse Primer | 5′-TACGGYTACCTTGTTACGACTT-3′ | SEQ ID NO: 3 |
| 1492R (used for | | |
| PCR) | | |
| Forward Primer | 5′-GGATTAGATACCCTGGTA-3′ | SEQ ID NO: 4 |
| 785F (used for | | |
| sequencing) | | |
| Reverse Primer | 5′-CCGTCAATTCMTTTRAGTTT-3′ | SEQ ID NO: 5 |
| 907R (used for | | |
| sequencing) | | |

As a result, it was confirmed that the isolated strain shows 99% homology to Sphingomonas paucimobilis. Accordingly, the strain was named “Sphingomonas paucimobilis GENSC03”. It was deposited with the Korea Research Institute of Bioscience and Biotechnology, a patented strain depository, on Mar. 11, 2021, and was given the accession number KCTC 14495BP.

### Example 2. Safety of Sphingomonas paucimobilis GENSC03

### Example 2-1. Confirmation of the Safety of the Strain

In order to confirm the safety of Sphingomonas paucimobilis GENSC03 strain for the human body, we confirmed the hemolytic activity which is the ability to destroy cells such as red blood cells present in the blood. The hemolytic activity can be confirmed through the formation of transparent rings when culturing the strain, and it is known that pathogenic microorganisms exhibit significant hemolytic activity.

Specifically, the Sphingomonas paucimobilis GENSC03 strain purely cultured in TSA medium was collected with an inoculation loop, streaked on a sheep blood agar, and then cultured aerobically at 37° C. for 48 hours. Afterwards, hemolysis was determined by observing whether a transparent ring was formed around the GENSC03 strain.

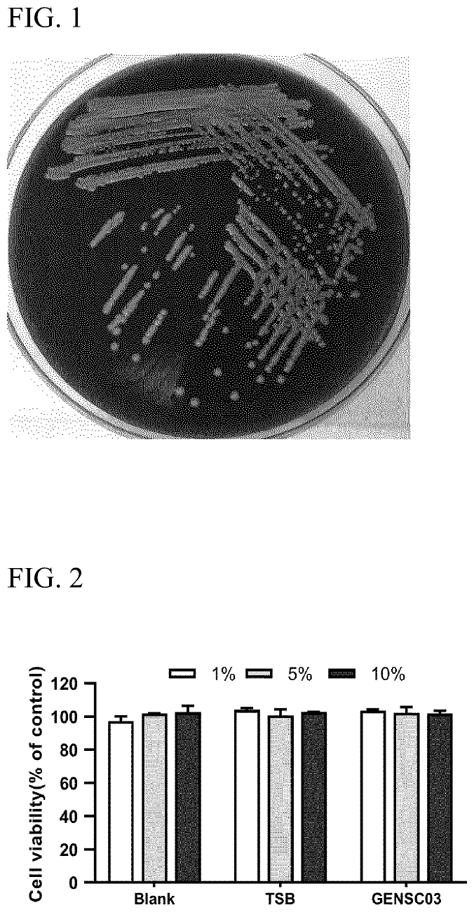

As a result, as shown in FIG. 1, it was confirmed that the GENSC03 strain cultured on blood agar medium was not hemolytic, as a transparent ring was not formed around the strain.

Through the above results, it was confirmed that the GENSC03 strain is harmless to the human body and has excellent safety, and thus it can be usefully used in cosmetics, medicines, and foods applied to the human body.

### Example 2-2. Confirmation of Cytotoxicity of Strain Culture

Through Example 2-1, it was confirmed that the Sphingomonas paucimobilis GENSC03 strain was not toxic to the human body. In this Example, we confirmed that the culture of the strain was also not toxic.

Specifically, the culture of the GENSC03 strain was prepared as follows. Sphingomonas paucimobilis GENSC03 strain was cultured aerobically on TSA solid plates at 30° C. for 24 hours. Thereafter, the single colony appearing on the solid plate was subcultured in 25 mL of Tryptic Soy Broth (TSB) liquid medium and cultured at 30° C. and 200 rpm for 24 hours. After 24 hours, 1% of the culture was inoculated into new TSB liquid medium and cultured under the same conditions (30° C., 200 rpm) for 24 hours. The supernatant was centrifuged and filtered through a 0.22 μm pore size filter.

Afterwards, the cytotoxicity of the culture was confirmed by the following method. First, human dermal fibroblasts were attached to a 96-well cell culture plate at 3×10⁴ cells/well for 24 hours. Then, the cultures were added at different concentrations (1, 5, 10 v/v %), and cultured for 24 hours under conditions of 5% CO₂ and 37° C. After completion of the culture, the cultured cell medium was removed, and washed with PBS. Then, 100 [symbol] of DMEM medium without FBS was added, and 10 [symbol] of EZ-Cytox reagent was further added and reacted for 2 hours. Absorbance (O.D) was measured at 570 nm with SpectraMax M2. Afterwards, the measured absorbance was converted to the formula “O.D sample/O.D control×100” to calculate the cell viability. From this, the cytotoxicity of the culture was determined.

As a result, as shown in FIG. 2, it was confirmed that the culture of the GENSC03 strain did not affect the cell viability of human dermal fibroblasts even at a concentration as high as 10%.

Through the above results, it was confirmed that the culture of the GENSC03 strain is not toxic to human cells, is harmless to the human body, and has excellent safety. Thus, it can be usefully used in cosmetics, medicines, and foods applied to the human body.

### Example 3. Skin Improvement Effect of Sphingomonas paucimobilis GENSC03 Strain Cultures

### Example 3-1. Confirmation of MMP-1 Expression Inhibition Effect

Through Example 2, it was confirmed that the Sphingomonas paucimobilis GENSC03 strain and its culture were safe when applied to the human body. In this Example, we tried to confirm the skin improvement effect for use as a cosmetic composition. First, in order to confirm the effects of preventing and improving skin wrinkles, enhancing skin elasticity, and strengthening the skin barrier, we confirmed the effect of suppressing the expression of MMP-1 (matrix metalloproteinase-1), an enzyme that decomposes collagen.

Specifically, human skin fibroblasts were dispensed in a 12-well plate at 2×10⁴ cells/well and cultured for 24 hours at 37° C. and 5% CO₂. After completion of culture, the cells were washed twice with PBS and then irradiated with 144 mJ/cm² of UVB using a UVB irradiator. Thereafter, the culture of the GENSC03 strain prepared by the method of Example 2-2 was added to DMEM medium without FBS at each concentration (0.1, 1, 5, 10 v/v %) and cultured. The control group not adding the culture of the GENSC03 strain was cultured for 72 hours in DMEM medium without FBS. After completion of culture, the supernatant of each well was obtained and the amount of expressed MMP-1 (pg/ml) was measured using an MMP-1 analysis ELISA kit. Each control group and experimental group were set as follows.
Control group: treated with TSB liquid medium
Positive control: treated with retinoic acid (1 μM)
Experimental group: treated with GENSC03 strain culture

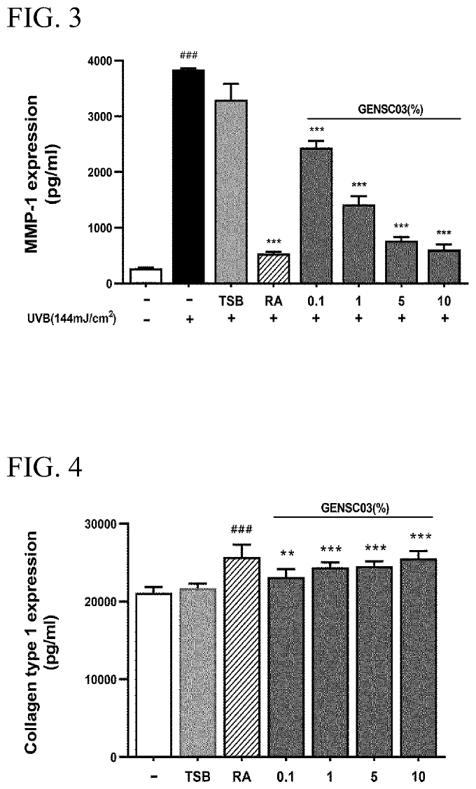

As a result, as shown in FIG. 3, when irradiated with UVB, MMP-1 is expressed at a high level of about 4,000 pg/ml. However, when treating with a culture of the GENSC03 strain, MMP-1 is expressed at a low level of about 500 to 2,500 pg/ml even though irradiated with UVB. Thus, it was confirmed that it was significantly suppressed by about 1.6 to 8 times or more. The effect of suppressing MMP-1 expression by the culture appears to be concentration-dependent. In particular, when treating with the culture at 10 v/v %, it shows an efficacy similar to that of retinoic acid, which is known to be excellent in suppressing MMP-1 expression or production.

Based on the above results, it was confirmed that the GENSC03 strain or its culture can suppress the expression of MMP-1, an enzyme that decomposes collagen, and thus exhibits excellent effects in preventing or improving skin wrinkles, and enhancing skin elasticity and strengthening the skin barrier.

### Example 3-2. Confirmation of Promoting the Expression of Collagen Type 1

As another experiment to confirm the effects of preventing and improving skin wrinkles, enhancing skin elasticity, and strengthening the skin barrier, we confirmed the effect of promoting the expression of collagen type 1.

Specifically, human skin fibroblasts were dispensed in a 12-well plate at 2×10⁴ cells/well and cultured for 24 hours at 37° C. and 5% CO₂. After completion of culture, the culture of the GENSC03 strain prepared by the method of Example 2-2 was added to DMEM medium without FBS at each concentration (0.1, 1, 5, 10 v/v %) and cultured. The control group not adding the culture of the GENSC03 strain was cultured for 72 hours in DMEM medium without FBS. After completion of culture, the supernatant of each well was obtained and the amount of expressed collagen type 1 (pg/ml) was measured using an collagen type 1 analysis ELISA kit. Each control group and experimental group were set as follows.
Control group: treated with TSB liquid medium
Positive control: treated with retinoic acid (1 μM)
Experimental group: treated with GENSC03 strain culture

As a result, as shown in FIG. 4, collagen type 1 with no treatment is expressed at a level of about 2,000 pg/ml. However, when treating with a culture of the GENSC03 strain, collagen type 1 is expressed at a high level of about 2,500 pg/ml and it was significantly increased by about 1.25 times or more. The effect of promoting collagen type 1 expression by the culture appears to be concentration-dependent. In particular, when treating with the culture at 10 v/v %, it shows an efficacy similar to that of retinoic acid (RA, 1 μM), which is known to be excellent in promoting collagen type 1 expression or production.

Based on the above results, it was confirmed that the GENSC03 strain or its culture can promote the expression and production of collagen, and thus exhibits excellent effects in preventing or improving skin wrinkles, and enhancing skin elasticity and strengthening the skin barrier.

### Example 3-3. Confirmation of Increased Expression of Filaggrin and Claudin-1

As an experiment to confirm the effects of preventing and improving skin wrinkles, enhancing skin elasticity, strengthening the skin barrier, and moisturizing the skin, we confirmed the effect of increasing the expression of filaggrin and claudin-1.

Specifically, human keratinocyte (HaCaT) were dispensed in a 6-well plate at 5×10⁵ cells/well and cultured for 24 hours at 37° C. and 5% CO₂. After completion of culture, the culture of the GENSC03 strain prepared by the method of Example 2-2 was added to DMEM medium without FBS at a concentration of 5 v/v % and cultured. The control group not adding the culture of the GENSC03 strain was cultured for 24 or 36 hours in DMEM medium without FBS. After culturing, the amount of filaggrin and claudin-1 mRNAs expressed in the cells was measured, and normalized to the mRNA expression level of β-actin. Each control group and experimental group were set as follows.
Control group: treated with TSB liquid medium
Positive control: treated with retinoic acid (1 μM)
Experimental group: treated with GENSC03 strain culture

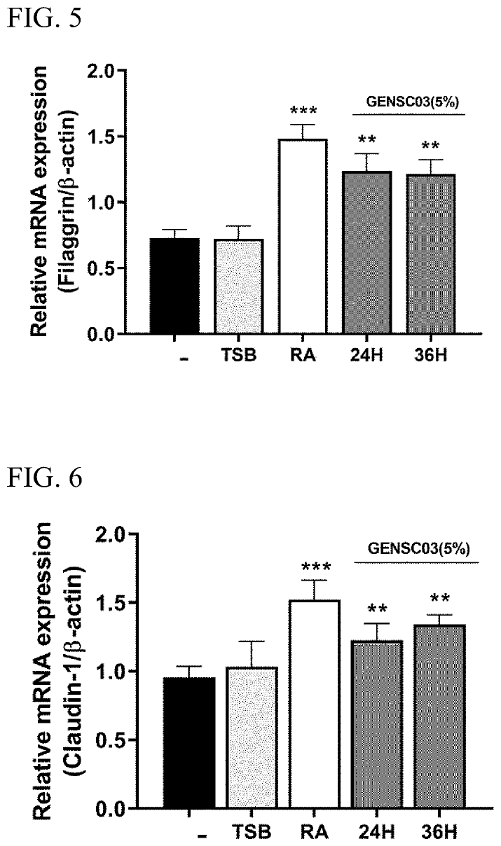

As a result, as shown in FIG. 5, when nothing is treated, filaggrin is expressed at a low level of about 0.75. However, when the culture of the GENSC03 strain is treated, it is expressed at a high level of about 1.25, which is a significant increase of more than about 1.5 times.

In addition, as shown in FIG. 6, when nothing is treated, claudin-1 is expressed at a low level of about 1.0. However, when the culture of the GENSC03 strain is treated, it is expressed at a high level of about 1.25 to 1.5, which is a significant increase of more than about 1.25 to 1.5 times.

From the above results, the GENSC03 strain or its culture can promote the production of filaggrin and claudin-1, which play a major role in maintaining skin moisture and strengthening the skin barrier, and thus has excellent skin moisturizing and skin barrier strengthening effects as well as skin elasticity enhancing effects.

### Deposit Information

Depository authority: Korea Research Institute of Bioscience and Biotechnology
Accession number: KCTC14495BP
Deposit date: Mar. 11, 2021

1. A Sphingomonas paucimobilis strain deposited with an accession number KCTC 14495BP.

2. The Sphingomonas paucimobilis strain according to claim 1, wherein the nucleotide sequence of the 16S rRNA gene of the strain is represented by SEQ ID NO: 1.

3. The Sphingomonas paucimobilis strain according to claim 1, wherein the strain decreases the expression or activity of Matrix metalloproteinase-1 (MMP-1).

4. The Sphingomonas paucimobilis strain according to claim 1, wherein the strain increases the expression or activity of one or more selected from the group consisting of collagen type 1, filaggrin and claudin-1.

5. A cosmetic composition comprising one or more selected from the group consisting of a Sphingomonas paucimobilis strain deposited with an accession number KCTC 14495BP, a culture of the strain, a fermentation product of the strain, a lysate of the strain, an extract of the strain and a cytoplasmic fraction obtained by lysing the strain.

6. The cosmetic composition according to claim 5, wherein the nucleotide sequence of the 16S rRNA gene of the Sphingomonas paucimobilis strain is represented by SEQ ID NO: 1.

7. The cosmetic composition according to claim 5, wherein the composition is used for preventing or improving skin wrinkles, enhancing skin elasticity, strengthening the skin barrier, or moisturizing the skin.

8. (canceled)

9. A method for preventing or improving skin wrinkles, enhancing skin elasticity, strengthening the skin barrier, or moisturizing the skin, comprising the step of administering to a subject one or more selected from the group consisting of a Sphingomonas paucimobilis strain deposited with an accession number KCTC 14495BP, a culture of the strain, a fermentation product of the strain, a lysate of the strain, an extract of the strain and a cytoplasmic fraction obtained by lysing the strain.


SEQUENCE LISTING (5 sequences)

SEQ ID NO: 1 - DNA, 1422 bp, Sphingomonas paucimobilis
  rRNA (1)..(1422): 16s rRNA gene of Sphingomonas paucimobilis GENSC03
ctgtcagaacgaacgctggcggcatgcctaacacatgcaagtcgaacgaaggcttcggccttagtggcgcacgggtgcgtaacgcgtgggaatctgcccttaggttcggaataacagctggaaacggctgctaataccggatgatatcgcgagatcaaagatttatcgcctgaggatgagcccgcgttggattaggtagttggtggggtaaaggcctaccaagccgacgatccatagctggtctgagaggatgatcagccacactgggactgagacacggcccagactcctacgggaggcagcagtggggaatattggacaatgggcgaaagcctgatccagcaatgccgcgtgagtgatgaaggccctagggttgtaaagctcttttacccgggaagataatgactgtaccgggagaataagccccggctaactccgtgccagcagccgcggtaatacggagggggctagcgttgttcggaattactgggcgtaaagcgcacgtaggcggctttgtaagtcagaggtgaaagcctggagctcaactccagaactgcctttgagactgcatcgcttgaatccaggagaggtcagtggaattccgagtgtagaggtgaaattcgtagatattcggaagaacaccagtggcgaaggcggctgactggactggtattgacgctgaggtgcgaaagcgtggggagcaaacaggattagataccctggtagtccacgccgtaaacgatgataactagctgtccgggcacttggtgcttgggtggcgcagctaacgcattaagttatccgcctggggagtacggccgcaaggttaaaactcaaaggaattgacgggggcctgcacaagcggtggagcatgtggtttaattcgaagcaacgcgcagaaccttaccagcgtttgacatggtaggacgacttccagagatggatttcttcccttcggggacctacacacaggtgctgcatggctgtcgtcagctcgtgtcgtgagatgttgggttaagtcccgcaacgagcgcaaccctcgcctttagttaccatcatttggttgggtactctaaaggaaccgccggtgataagccggaggaaggtggggatgacgtcaagtcctcatggcccttacgcgctgggctacacacgtgctacaatggcaactacagtgggcagcgaccctgcgagggcgagctaatccccaaaagttgtctcagttcggattgttctctgcaactcgagagcatgaaggcggaatcgctagtaatcgcggatcagcatgccgcggtgaatacgttcccaggccttgtacacaccgcccgtcacaccatgggagttggattcacccgaaggcgttgcgccaacctagcaataggaagcaggcgaccacggtgggttcagcgactggggtgaagc

SEQ ID NO: 2 - DNA, 20 bp, Artificial Sequence
  27F
agagtttgatcmtggctcag

SEQ ID NO: 3 - DNA, 22 bp, Artificial Sequence
  1492R
tacggytaccttgttacgactt

SEQ ID NO: 4 - DNA, 18 bp, Artificial Sequence
  785F
ggattagataccctggta

SEQ ID NO: 5 - DNA, 20 bp, Artificial Sequence
  907R
ccgtcaattcmtttragttt